(19) United States
(12) Patent Application Publication
(10) Pub. No.: US 2006/0247596 A1
(43) Pub. Date: Nov. 2, 2006

(54) Absorbent article having front and back ears

(75) Inventor: Wendy L. Van Dyke, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/116,654

(22) Filed: Apr. 27, 2005

(51) Int. Cl. A61F 13/15

(52) U.S. Cl. 604/391; 604/385.01

(57) ABSTRACT

Disclosed is a disposable absorbent article including a liquid impermeable outercover, an absorbent body disposed on the outercover and a pair of front ears and a pair of back ears.

### BACKGROUND OF THE INVENTION

The present invention relates to disposable absorbent articles. More specifically, the invention relates to a disposable absorbent article, such as a disposable diaper, that includes a pair of front ears and a pair of back ears.

Absorbent articles such as disposable diapers are useful to absorb and contain body wastes. In certain aspects, these products can include a pair of ears extending from the article in the back waist region. In addition, these articles can also include a pair of ears extending from the article in the front waist region. The ears can be useful for providing better hip coverage to the wearer. Further, the ears can be stretchable or even elastomeric for improved fit and comfort. Moreover, the ears can include fastener material to keep the article about the hips and waist of the wearer.

While absorbent articles that include both front and back ears may be advantageous for purposes of fastening and aesthetics, adding components to the product can increase material costs and can also increase the complexity of the manufacturing process. That is, one system may be necessary to supply and apply the back ears on the article, while a separate manufacturing system may be necessary to supply and apply the front ears on the article. In such a configuration, the front ears and back ears would, in some instances, have to be initially separated from separate webs of material.

Thus, there is a need for an absorbent article having front and back ears that are configured to come from a single process and initially from a single web of material. Further, there is a need for an article with front ears having a complementary shape and back ears having a complementary shape to reduce trim waste. Still further there is a need for an article with front and back ears that have different shapes, but that are still configured to be produced from a single process and web of material.

### SUMMARY OF THE INVENTION

In one aspect, the present invention is directed to a disposable absorbent article defining a lateral direction, a longitudinal direction, a front waist region, a rear waist region, a crotch region extending between and connecting the waist regions, an interior surface and an exterior surface. The absorbent article includes a liquid impermeable outercover, an absorbent body disposed on the interior surface of the article relative to the outercover, and pair of front ears joined to the article in the front waist region. Each of the front ears defines a front ear proximal edge having a front ear proximal edge length and each of the front ears includes a front ear base material. The article also includes a pair of back ears joined to the article in the back waist region, each of the back ears defining a back ear proximal edge having a back ear proximal edge length. Each of the back ears further includes a back ear base material and fastener material attached to the back ear base material, where the front ear proximal edge length is substantially the same as the back ear proximal edge length.

In another aspect, the present invention is directed to a disposable absorbent article defining a lateral direction, a longitudinal direction, a front waist region, a rear waist region, a crotch region extending between and connecting the waist regions, an interior surface and an exterior surface. The absorbent article includes a liquid impermeable outercover, an absorbent body disposed on the interior surface of the article relative to the outercover, and a pair of front ears joined to the article in the front waist region. Each of the front ears defines a front ear proximal edge having a front ear proximal edge length and each of the front ears includes a front ear base material and fastener material attached to the front ear base material. The fastener also includes a pair of back ears joined to the article in the back waist region, each of the back ears defining a back ear proximal edge having a back ear proximal edge length. Each of the back ears includes a back ear base material and fastener material attached to the back ear base material where the front ear proximal edge length is substantially the same as the back ear proximal edge length.

In yet another aspect, the present invention is directed to a disposable absorbent article defining a lateral direction, a longitudinal direction, a front waist region, a rear waist region, a crotch region extending between and connecting the waist regions, an interior surface and an exterior surface. The absorbent article includes a liquid impermeable outercover an absorbent body disposed on the interior surface of the article relative to the outercover and a pair of front ears joined to the article in the front waist region. Each of the front ears defines a front ear proximal edge having a front ear proximal edge length. The article also includes a pair of back ears joined to the article in the back waist region. Each of the back ears defines a back ear proximal edge having a back ear proximal edge length where the front ear proximal edge length is substantially the same as the back ear proximal edge length. In addition, the front ears have a complementary shape with each other and the back ears have a complementary shape with each other, and the front ears and the back ears have different shapes.

The above-mentioned and other aspects of the present invention will become more apparent, and the invention itself will be better understood by reference to the drawings and the following description of the drawings.

### BRIEF DESCRIPTION OF THE DRAWINGS

FIG. 1 representatively illustrates a side view of a diaper of the present invention with a mechanical fastening system the diaper in a fastened configuration;

FIG. 2 representatively illustrates a plan view of a diaper similar to that of FIG. 1 in an unfastened, stretched and laid flat condition, and showing the surface of the diaper that faces the wearer with portions cut away to show underlying features;

FIG. 3 representatively illustrates a plan view similar to FIG. 2, but showing the surface of the diaper that faces away from the wearer when worn, and with portions cut away to show underlying features; and

FIGS. 4 and 5 representatively illustrate examples of different ears of the present invention.

Corresponding reference characters indicate corresponding parts throughout the drawings.

### DEFINITIONS

Within the context of this specification, each term or phrase below includes the following meaning or meanings:

“Attach” and its derivatives refer to the adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Connect” and its derivatives refer to the adhering, bonding, attaching, sewing together, or the like, of two elements. Two elements will be considered to be connected together when they are integral with one another or connected directly to one another or indirectly to one another, such as when each is directly connected to intermediate elements. “Connect” and its derivatives include permanent, releasable, or refastenable connection.

“Complementary shape” when used in the context to describe ears refers to the configuration of the ear allowing it to be nested with another ear with a minimum of gapping between the ears such that if the two ears were to be cut from a web of material there would not be any trim waste material resulting from spacing between the ears.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 25 percent (to 125 percent) of its relaxed length and will recover, upon release of the applied force, at least 40 percent of its elongation.

“Extensible” refers to a material or composite which is capable of extension or deformation without breaking, but does not substantially recover its original size and shape after removal of a force causing the extension or deformation. Suitably, an extensible material or composite can be elongated by at least 25 percent (to 125 percent) of its relaxed length.

“Fiber” refers to a continuous or discontinuous member having a high ratio of length to diameter or width. Thus, a fiber may be a filament, a thread, a strand, a yarn, or any other member or combination of these members.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Join” and its derivatives refer to the adhering, bonding, sewing together, or the like, of two separate elements. Two elements will be considered to be joined together when they are joined directly to one another or indirectly to one another, such as when each is directly joined to intermediate elements. “Join” and its derivatives include permanent, releasable, or refastenable joinder.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Stretchable” means that a material can be stretched, without breaking, by at least 25 percent (to 125 percent of its initial (unstretched) length) in at least one direction. Elastic materials and extensible materials are each stretchable materials.

“superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride.

These terms may be defined with additional language in the remaining portions of the specification.

### DETAILED DESCRIPTION

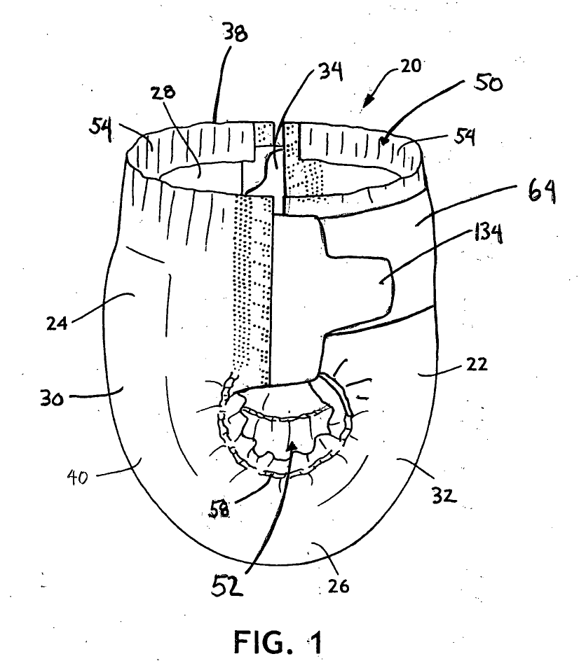

Referring now to the drawings and in particular to FIG. 1, an absorbent article of the present invention is representatively illustrated in the form of baby diaper and is indicated in its entirety by the reference numeral 20. The diaper 20 includes a pair of front ears 34 and a pair of back ears 134. The diaper 20 may suitably be disposable, which refers to articles that are intended to be discarded after a limited period of use instead of being laundered or otherwise conditioned for reuse. It should also be understood that the present invention is suitable for use with various other absorbent articles intended for personal wear, including but not limited to children's training pants, feminine hygiene products, incontinence products, medical garments, surgical pads and bandages, other personal care or health care garments, and the like without departing from the scope of the present invention.

By way of illustration only, various materials and methods for constructing diapers such as the diapers 20 of the various aspects of the present invention are disclosed in Application docket number 20,210; U.S. Pat. No. 5,496,298 issued Mar. 5, 1996 to Kuepper et al.; U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996, to Hanson et al., each of which are incorporated herein by reference to the extent that they are consistent (i.e., not in conflict) herewith.

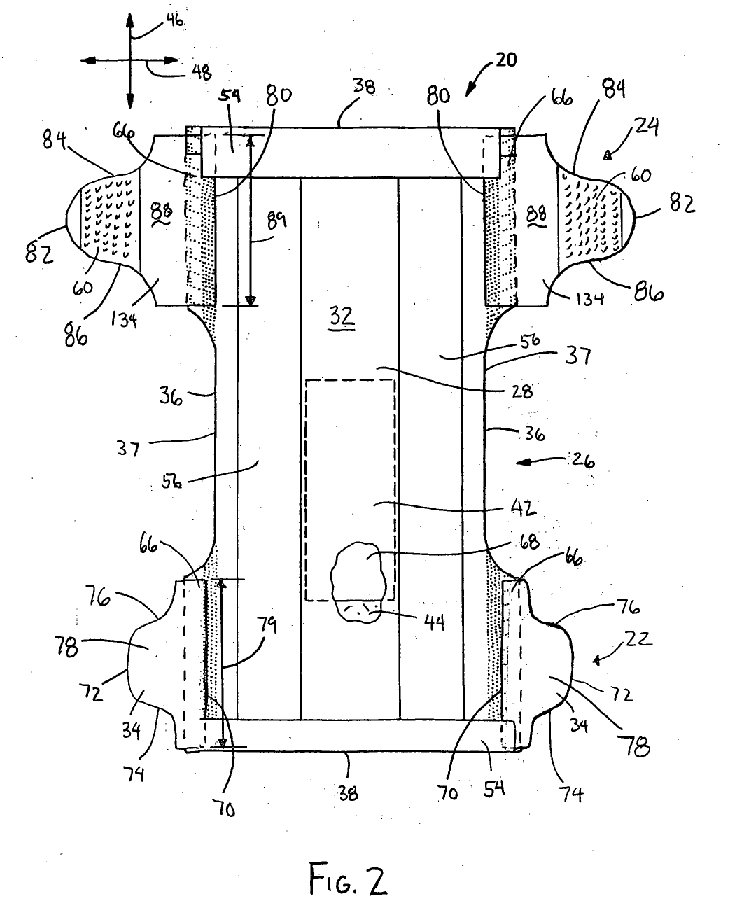

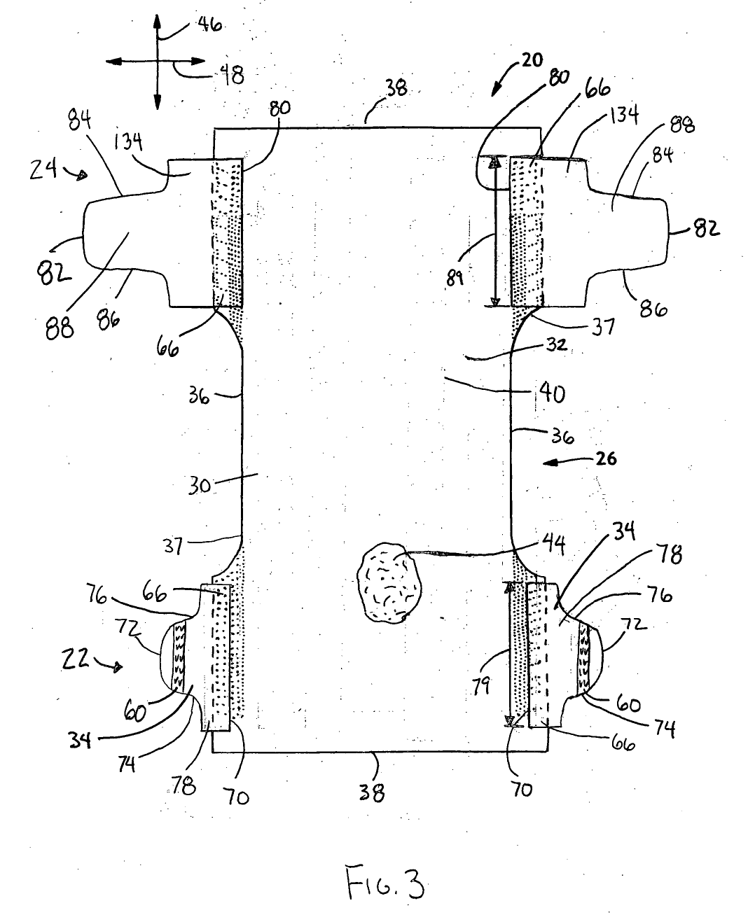

The diaper 20 is illustrated in FIG. 1 in a fastened condition. The diaper 20 defines a longitudinal direction 46 and a lateral direction 48 perpendicular to the longitudinal direction as shown in FIGS. 2 and 3. The diaper 20 further defines a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The front and back waist regions 22, 24 includes those portions of the diaper 20, which when worn, wholly or partially cover or encircle the waist or mid-lower torso of the wearer. The crotch region 26 generally is that portion of the diaper 20 which, when worn, is positioned between the legs of the wearer and covers the lower torso and crotch of the wearer. The diaper 20 also define an inner surface 28 adapted in use to be disposed toward the wearer, and an outer surface 30 opposite the inner surface. With additional reference to FIGS. 2 and 3, the diaper 20 has a pair of laterally opposite side edges 36 and a pair of longitudinally opposite waist edges 38.

The illustrated diaper 20 can include an absorbent assembly, generally indicated at 32 defining a pair of laterally opposed side margins 37. For example, in the aspect of FIGS. 1-3, the diaper 20 includes a generally rectangular absorbent assembly 32 and ears 34, 134 formed separately from and secured to the absorbent assembly. The ears 34, 134 can be joined along seams 66 to the absorbent assembly 32 in the respective front and back waist regions 22 and 24 of the diaper 20. More particularly, the pair of front ears 34 can be permanently joined to and extend laterally outward from the absorbent assembly 32 at the front waist region 22, and the pair of back ears 134 can be permanently joined to and extend laterally from the absorbent assembly 32 at the back waist region 24. The ears 34 and 134 may be joined to the absorbent assembly 32 using attachment means known to those skilled in the art such as adhesive, thermal bonding, pressure bonding, ultrasonic bonding, and the like or combinations thereof.

The absorbent assembly 32 is illustrated in FIGS. 1-3 as having a substantially rectangular shape. However, it is contemplated that the absorbent assembly 32 may have other shapes (e.g., hourglass, T-shaped, τ-shaped, and the like) without departing from the scope of this invention.

The absorbent assembly 32 includes an outercover 40 and a bodyside liner 42 (FIG. 2) in a superposed relation therewith. The liner 42 can be suitably joined to the outercover 40 along at least a portion of the longitudinal ends of the diaper 20. The liner 42 can be suitably adapted, i.e., positioned relative to the other components of the diaper 20, to contact the wearer's skin during wear of the diaper. The absorbent assembly 32 also includes an absorbent body 44 (FIG. 3) disposed on the interior surface of the article relative to the outercover 40 for absorbing liquid body exudates. For example, the absorbent body 44 can be located between the outercover 40 and the bodyside liner 42. The liner 42 can be suitably joined to the outercover 40 along at least a portion of the longitudinal ends of the diaper 20. The bodyside liner 42 and the outercover 40 can, for example, be attached to each other by adhesive, ultrasonic bonding, thermal bonding or by other suitable attachment techniques known in the art. Moreover, at least a portion of the absorbent body 44 can optionally be attached to the bodyside liner 42 and/or the outercover 40 utilizing the methods described above.

The diaper 20 may further include a pair of containment flaps 56 for inhibiting the lateral flow of body exudates. As illustrated in FIG. 2, the containment flaps 56 can be operatively attached to the diaper 20 in any suitable manner as is well known in the art. In particular, suitable constructions and arrangements for the containment flaps 56 are generally well known to those skilled in the art and are described in U.S. Pat. No. 4,704,116 issued Nov. 3, 1987 to Enloe, which is incorporated herein by reference to the extent that it is consistent (i.e., not in conflict) herewith.

To further enhance containment and/or absorption of body exudates, the diaper 20 may include waist elastic members 54 in the front and/or back waist regions 22 and 24 of the diaper 20. Likewise, the diaper 20 may include leg elastic members 58, as are known to those skilled in the art. The waist elastic members 54 and the leg elastic members 58 can be formed of any suitable elastic material that is well known to those skilled in the art. For example, suitable elastic materials include sheets, strands or ribbons of natural rubber, synthetic rubber, or thermoplastic elastomeric polymers. In one aspect of the invention, the waist elastics and/or the leg elastics may include a plurality of dry-spun coalesced multi-filament spandex elastomeric threads sold under the trade name LYCRA and available from Invista of Wilmington, Del., U.S.A.

The outercover 40 may suitably include a material that is substantially liquid impermeable. The outercover 40 may be provided by a single layer of liquid impermeable material, or more suitably include a multi-layered laminate structure in which at least one of the layers is liquid impermeable. In particular aspects, the outer layer may suitably provide a relatively cloth-like texture to the wearer. A suitable liquid impermeable film for use as a liquid impermeable inner layer, or a single layer liquid impermeable outercover 40 is a 0.025 millimeter (1.0 mil) polyethylene film commercially available from Edison Plastics Company of South Plainfield, N.J. Alternatively, the outercover 40 may include a woven or non-woven fibrous web layer that has been totally or partially constructed or treated to impart the desired levels of liquid impermeability to selected regions that are adjacent or proximate the absorbent body.

The outercover 40 may also be stretchable, and in some aspects it may be elastomeric. For example, such an outercover material can include a 0.3 osy polypropylene spunbond that is necked 60 percent in the lateral direction 40 and creped 60 percent in the longitudinal direction 48, laminated with 3 grams per square meter (gsm) Bostik-Findley H2525A styrene-isoprene-styrene based adhesive to 8 gsm PEBAX 2533 film with 20 percent TiO₂ concentrate. Reference is made to U.S. Pat. No. 5,883,028, issued to Morman et al., U.S. Pat. No. 5,116,662 issued to Morman and U.S. Pat. No. 5,114,781 issued to Morman, all of which are hereby incorporated herein by reference, for additional information regarding suitable outercover materials.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent body 44. A suitable liquid permeable bodyside liner 42 is a nonwoven polyethylene/polypropylene bicomponent web having a basis weight of about 27 gsm; the web may be spunbonded or a bonded carded web. Optionally, the bodyside liner 42 may be treated with a surfactant to increase the wettability of the liner material.

Alternatively, the bodyside liner 42 may also be stretchable, and in some aspects it may be elastomeric. For instance, the liner 42 can be a non-woven, spunbond polypropylene fabric composed of about 2 to 3 denier fibers formed into a web having a basis weight of about 12 gsm which is necked approximately 60 percent. Strands of about 9 gsm KRATON G2760 elastomer material placed eight strands per inch (2.54 cm) can be adhered to the necked spunbond material to impart elasticity to the spunbond fabric. The fabric can be surface treated with an operative amount of surfactant, such as about 0.6 percent AHCOVEL Base N62 surfactant, available from ICI Americas, a business having offices in Wilmington, Del., U.S.A. Other suitable materials may be extensible biaxially stretchable materials, such as a neck stretched/creped spunbond. Reference is made to U.S. Pat. No. 6,552,245, issued Apr. 22, 2003, to Roessler et al., which is incorporated by reference herein to the extent that it is consistent (i.e., not in conflict) herewith.

The absorbent body 44 is suitably compressible, conformable and capable of absorbing and retaining liquid body exudates released by the wearer. For example, the absorbent assembly can include a matrix of absorbent fibers, and more suitably cellulosic fluff, such as wood pulp fluff, and superabsorbent particles. One suitable pulp fluff is identified with the trade designation CR1654, commercially available from U.S. Alliance, Childersburg, Ala., U.S.A. As an alternative to wood pulp fluff, synthetic fibers, polymeric fibers, meltblown fibers, short cut homofil bicomponent synthetic fibers, or other natural fibers may be used. Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid. Suitable superabsorbent materials are available from various commercial vendors, such as Dow Chemical Company of Midland, Mich., U.S.A., and Stockhausen Inc., Greensboro, N.C., U.S.A.

The absorbent body 44 can be in a variety of shapes and configurations as are known in the art, such as rectangular, hourglass shaped, τ-shaped, and the like. In addition, the absorbent body 44 can have a density within the range of about 0.10 to about 0.5 grams per cubic centimeter and may be wrapped or encompassed by a suitable tissue or nonwoven wrap for maintaining the integrity and/or the shape of the absorbent assembly.

In one aspect, the absorbent body 44 may be stretchable so as not to inhibit the stretchability of other components to which the absorbent body may be adhered, such as the outercover 40 and/or the bodyside liner 42. For example, the absorbent body may include materials disclosed in U.S. Pat. Nos. 5,964,743, 5,645,542, 6,231,557, 6,362,389, and international patent application WO 03/051254, the disclosure of each of which is incorporated by reference herein.

In some aspects, a surge management layer 68 may be included in the diaper 20. The surge management layer 68 may be positioned in the diaper 20 in a variety of locations as is known in the art. For example, the surge management layer 68 can be proximate the absorbent body 44, for example between the absorbent body 44 and the bodyside liner 22, and attached to one or more components of the diaper 20 by methods known in the art, such as by adhesive, ultrasonic bonding, pressure bonding, thermal bonding, and the like or combinations thereof.

A surge management layer helps to decelerate and diffuse surges or gushes of liquid that may be rapidly introduced into the absorbent body 44. Desirably, the surge management layer 68 can rapidly accept and temporarily hold the liquid prior to releasing the liquid into the storage or retention portions of the absorbent body 44. Examples of suitable surge management layers are described in U.S. Pat. No. 5,486,166 and U.S. Pat. No. 5,490,846, the contents of which are incorporated herein by reference to the extent that they are consistent (i.e., not in conflict) herewith.

As mentioned above, the various aspects of the diaper 20 of the present invention can also include a pair of front ears 34 and a pair of back ears 134. The front ears 34 include a front ear proximal edge 70, an opposed front ear distal edge 72, a front ear first connecting edge 74 and a front ear second connecting edge 76. The back ears 134 include a back ear proximal edge 80, an opposed back ear distal edge 82, a back ear first connecting edge 84 and a back ear second connecting edge 86.

The proximal edges 70, 80 are the portion of the ears 34, 134 that are joined to the side margins 37 of the absorbent assembly 32 in the respective waist regions 22, 24. The distal edges 72, 82 are those edges of the ears 34 and 134 that are opposite the proximal edges 70, 80 moving in a direction outboard from the absorbent assembly 32. The first and second connecting edges 74, 76, 84, 86 connect the proximal edges 70, 80 and the distal edges 72, 82 of the ears 34 and 134.

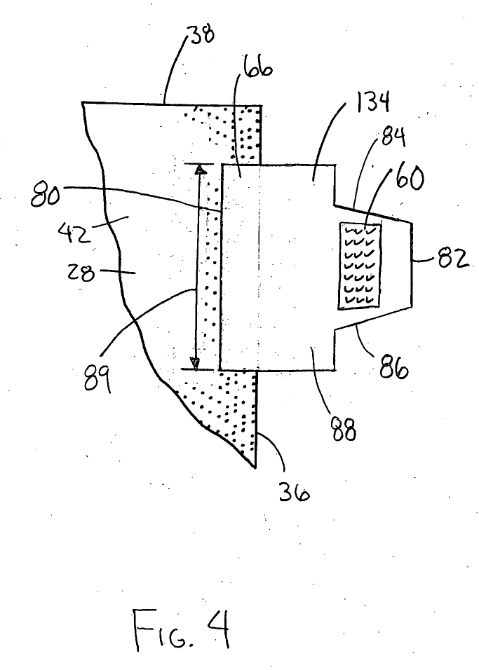

The ears 34 and 134 may be joined to the absorbent assembly 32 in a variety of ways as are known in the art. For example, the ears 34, 134 may be joined to the absorbent assembly 32 with adhesive, ultrasonic bonds, pressure bonds, thermal bonds, and the like, or combinations thereof. In addition, the ears 34 and 134 may be joined to the absorbent assembly 32 on the inner surface 28 of the diaper 20 (FIGS. 2, 4 and 5), the outer surface 30 of the diaper 20 (FIG. 3), or can be sandwiched between at least some of the layers that can make up the absorbent assembly 32 (FIG. 1), such as the outercover 40 and liner 42. Moreover, the ears 34, 134 may be attached in various combinations. For example, the front ears 34 can be joined to the absorbent assembly 32 on the inner surface 28 of the diaper 20 while the back ears 134 can be joined to the absorbent assembly 32 on the outer surface 30 of the diaper 20.

In particular aspects, the front ears 34 can include a front ear base material 78 and the back ears can include a back ear base material 88. The base materials 78, 88 may be provided by materials as are known in the art such as woven materials, nonwoven materials, or combinations thereof. In a particular aspect, at least a portion of the base materials 78, 88 are an elastomeric material capable of elongating in at least the lateral direction 40. Examples of a suitable elastomeric material for use in connection with the base materials 78, 88 are a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference in their entirety to the extent they are consistent (i.e., not in conflict) herewith. As is known in the art, the base materials 78, 88 can optionally include nonstretchable materials or stretchable but inelastic materials. For example, the base material 78, 88 can include various nonstretchable nonwovens such as a spunponded material or a spunbond/meltblown/spunbond (SMS) material. Alternatively, the base materials 78, 88 can include film materials that could also be suitable for use in connection with the outercover 40.

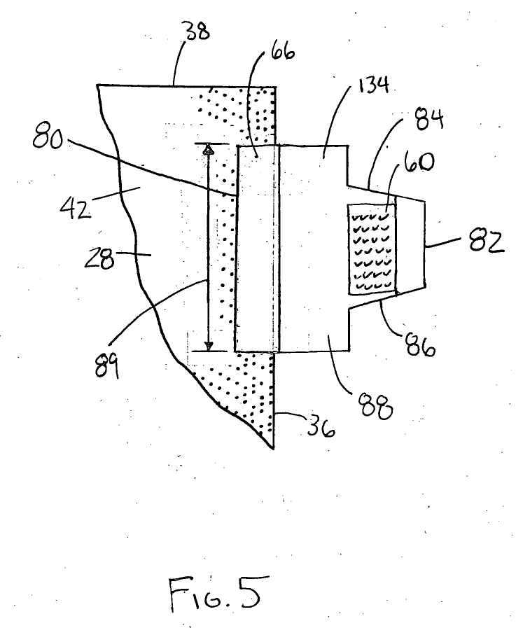

In yet another alternative, the base materials 78, 88 can include a combination of stretchable and nonstretchable materials. For example, as representatively illustrated in FIG. 5 the base material 88 adjacent the distal edge 82 and the proximal edge 80 could be nonstretchable materials while the base material 88 between the edges 80, 82 could be stretchable. While FIG. 5 illustrates a back ear 134 specifically, it can be readily appreciated by one of ordinary skill in the art that such a configuration can be utilized with a front ear 34 as well.

The component layers making up the base materials 78 and 88 may be assembled in various ways as are known in the art. For example, the layers in the base materials 78, 88 can be assembled with adhesives, ultrasonic bonding, pressure bonding, thermal bonding, and the like or combinations thereof. Optionally, the base materials 78, 88 can be provided by a single layer of material.

The front ear base material 78 and the back ear base material 88 can optionally be provided by the same materials or combination of materials. For example, the front ear base material 78 and the back ear base material 88 may be provided by a uniform web of material or by a web of material having the same components in the portion providing the back ear base material 88 as in the portion providing the front ear base material. As such, the back ear base material 88 and the front ear base material 78 can include materials that have substantially the same basis weight. By “substantially the same basis weight” it is meant that the basis weight of at least a portion of the material providing the front ear base material 78 is within 2% of at least a portion of the material providing the back ear base material 88. In addition, the at least a portion of the back ear base material 88 and at least a portion of the front ear base material 78 can be the same type of materials. That is, at least a portion of the front ear base material 78 and at least a portion of the back ear base material 88 could each be SMS, or NBL, or any other material that may suitably be used for the front and back ear base materials 78, 88. Still further, the back ear base material 88 and the front ear base material 78 can be provided by the identical material or combination of materials.

In yet another alternative, the back ear base material 88 and the front ear base material 78 may be different, but they nonetheless can optionally be provided by the same source web of material. For example, the web may have different component layers in the portions that provide the front ear base material 78 than in the portion that provides the back ear base material 88.

Suitable source webs for the ears 34, 134 are described in U.S. patent application Ser. No. ______, entitled COMPOSITE WEB filed in the name of Van Dyke concurrently with the present application, the entire disclosure of which is hereby incorporated by reference to the extent that it is consistent (i.e., not in conflict) herewith.

The proximal edges 70, 80 of the ears of the various aspects of the present invention can further define a proximal edge length. In particular, the back ear proximal edges 80 can define a back ear proximal edge length, indicated by the arrow marked 89, and the front ear proximal edges 70 can define a front ear proximal edge length, indicated by the arrow marked 79. The front ear proximal edge length 79 can be substantially the same as the back ear proximal edge length 89. By “substantially the same proximal edge length” it is meant that the front ear proximal edge length 79 is within 2% of the back ear proximal edge length 89. Suitably, the front ear proximal edge length 79 can be equal to the back ear proximal edge length 89. In such an aspect, the both the front ears 34 and the back ears 134 can be provided in process from the same web of material. In particular, since the proximal edge length 79, 89 of the ears 34, 134 are substantially the same, the front ears 34 and the back ears 134 have the same repeat length. As such, as the diaper 20 is being converted, the ears 34 and 134 can all be separated from the same web simultaneously thereby increasing process efficiency and reducing process equipment and material costs.

The ears 34 and 134 can further have a variety of shapes as are known in the art. For example, as representatively illustrated in FIGS. 2 and 3, the back ears 134 can have a complementary shape with each other. Similarly, front ears 34 can also have a complementary shape with each other. Moreover, as the front ears 34 can have complementary shapes with each other, and the back ears 134 can simultaneously have complementary shapes with each other, but the front ears 34 can have a different shape than the back ears 134 (FIGS. 2 and 3). As such, in configurations where the ears 34 and 134 also feature substantially the same proximal edge length, the ears 34 and 134 may all be process from a single source web at the same time, but still have individual shapes. This may be desirable as the front ears 34 may have a different purpose than the back ears 134, or it may be desirable from an aesthetic or material cost perspective. Alternatively, the front ears 34 and back ears 134 may optionally have a substantially similar shape.

As representatively illustrated in FIGS. 2-5, the front and back ears 34 and 134 can also optionally include fastener material 60. For example, one of or each of the back ears 134 can include fastener material 60 attached to the back ear base material 88. Optionally, one of or each of the front ears 34 can include fastener material 60 attached to the front ear base material 78. Moreover, the back ears 134 can include fastener material 60 while the front ears 34 do not, or vice versa. Still further, all of the ears 34 and 134 can include fastener material 60.

The fastener material 60 may include any refastenable fasteners suitable for absorbent articles, such as adhesive fasteners, cohesive fasteners, mechanical fasteners, or the like. In one aspect of the invention, the fastener material 60 is active fastener materials for improved performance. Suitable active fastener materials are interlocking geometric-shaped materials that are intended to engage another material such as hooks, bulbs, mushrooms, arrowheads, balls on stems, male mating components or the like. Accordingly, the fastener material 60 attached to the back ear base material 88 can be active fastener material. Likewise, the fastener material 60 attached to the front ear base material 78 can be active fastener material. One specific example of a fastener material is VELCRO HTH 858 or VELCRO HTH 823 available from Velcro Industries B.V., Amsterdam, Netherlands.

The fastener material 60 may be arranged on the ears 34, 134 to engage different portions of the diaper 20. For example, the fastener material 60 attached to the back ears 134 can be configured to engage the outer surface 30 of the diaper 20. In such a configuration, the fastener material 60 is attached to the back ear 134 on the inner surface 28 of the diaper 20. Further, the fastener material 60 attached to the front ear 34 can be configured to engage the inner surface 28 of the diaper 20. In such a configuration, the fastener material 60 is attached to the front ear 34 on the outer surface 30 of the diaper 20. Accordingly, the fastener material 60 on the ears 34, 134 may be configured to refastenably engage directly with the outercover 40 or the liner 42 of the diaper 20. Alternatively, at least one attachment panel 64 may be suitably located on the diaper 20 to which the fastener material 60 on the ears 34, 134 is configured to engage. For example, as representatively illustrated in FIG. 1, an attachment panel 64 can be located on the outercover 40.

Thus, the fastener material 60 can be employed to attach the back waist region 24 of the diaper 20 to the front waist region 22 of the diaper 20 about the waist of the wearer. Accordingly, the waist edges 38 of the diaper 20 are configured to encircle the waist of the wearer to define the waist opening 50 of the diaper, while the side edges 36 of the diaper 20 define the leg openings 52 (FIG. 1).

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A disposable absorbent article defining a lateral direction, a longitudinal direction, a front waist region, a rear waist region, a crotch region extending between and connecting said waist regions, an interior surface, an exterior surface, said absorbent article comprising:
A liquid impermeable outercover;
An absorbent body disposed on said interior surface of said article relative to said outercover;
A pair of front ears joined to said article in said front waist region, each of said front ears defining a front ear proximal edge having a front ear proximal edge length, and each of said front ears comprising a front ear base material;
A pair of back ears joined to said article in said back waist region, each of said back ears defining a back ear proximal edge having a back ear proximal edge length and each of said back ears comprising a back ear base material and fastener material attached to said back ear base material; wherein
Said front ear proximal edge length is substantially the same as said back ear proximal edge length.

2. The article of claim 1 wherein said front ear proximal edge length is equal to said back ear proximal edge length.

3. The absorbent article of claim 1 wherein at least a portion of said back ear base material and at least a portion of said front ear base material are substantially the same basis weight.

4. The absorbent article of claim 1 wherein at least a portion of said back ear base material and at least a portion of said front ear base material are substantially the same type of materials.

5. The absorbent article of claim 1 wherein said back ear base material and said front ear base material are provided by identical materials.

6. The absorbent article of claim 1 wherein said front ears comprise fastener material attached to said front ear base material.

7. The absorbent article of claim 6 wherein said fastener material attached to said back ears is configured to engage the exterior surface of said article and said fastener material attached to said front ears is configured to engage said interior surface of said article.

8. The absorbent article of claim 1 wherein said fastener material attached to said front ear base material is active fastener material.

9. The absorbent article of claim 1 wherein said fastener material attached to said back ear base material is active fastener material.

10. The absorbent article of claim 1 wherein said back ears are elastomeric and may be elongated in said lateral direction.

11. The absorbent article of claim 1 wherein said front ears have a complementary shape with each other.

12. The absorbent article of claim 1 wherein said back ears have a complementary shape with each other.

13. The absorbent article of claim 1 wherein said fastener material attached to said front ears and said back ears is hook material.

14. The absorbent article of claim 1 wherein said front ears have a complementary shape with each other and said back ears have a complementary shape with each other.

15. The absorbent article of claim 14 wherein said front ears and said back ears have different shapes.

16. A disposable absorbent article defining a lateral direction, a longitudinal direction, a front waist region, a rear waist region, a crotch region extending between and connecting said waist regions, an interior surface, an exterior surface, said absorbent article comprising:
A liquid impermeable outercover;
An absorbent body disposed on said interior surface of said article relative to said outercover;
A pair of front ears joined to said article in said front waist region, each of said front ears defining a front ear proximal edge having a front ear proximal edge length and each of said front ears comprising a front ear base material and fastener material attached to said front ear base material;
A pair of back ears joined to said article in said back waist region, each of said back ears defining a back ear proximal edge having a back ear proximal edge length and each of said back ears comprising a back ear base material and fastener material attached to said back ear base material; wherein
Said front ear proximal edge length is substantially the same as said back ear proximal edge length.

17. The disposable absorbent article of claim 16 wherein each of said back ears are elastomeric.

18. The article of claim 16 wherein said front ear proximal edge length is equal to said back ear proximal edge length.

19. The absorbent article of claim 16 wherein said front ears have a complementary shape with each other and said back ears have a complementary shape with each other.

20. The absorbent article of claim 19 wherein said front ears and said back ears have different shapes.

21. A disposable absorbent article defining a lateral direction, a longitudinal direction, a front waist region, a rear waist region, a crotch region extending between and connecting said waist regions, an interior surface, an exterior surface, said absorbent article comprising:
A liquid impermeable outercover;
An absorbent body disposed on said interior surface of said article relative to said outercover;
A pair of front ears joined to said article in said front waist region, each of said front ears defining a front ear proximal edge having a front ear proximal edge length;
A pair of back ears joined to said article in said back waist region, each of said back ears defining a back ear proximal edge having a back ear proximal edge length; wherein
Said front ear proximal edge length is substantially the same as said back ear proximal edge length, said front ears have a complementary shape with each other and said back ears have a complementary shape with each other, and said front ears and said back ears have different shapes.
